(19) United States
(12) Patent Application Publication
(10) Pub. No.: US 2022/0183785 A1
(43) Pub. Date: Jun. 16, 2022

(54) Neuro-Surgical Clamp

(71) Applicant: Jeffrey Alan Greenberg, Reston, VA (US)

(72) Inventor: Jeffrey Alan Greenberg, Reston, VA (US)

(21) Appl. No.: 17/397,695

(22) Filed: Aug. 9, 2021

Related U.S. Application Data
(63) Continuation of application No. 16/289,131, filed on Feb. 28, 2019, now abandoned.
Application No. 16/289,131 is a continuation of application No. 15/729,245, filed on Oct. 10, 2017, now abandoned.
Application No. 15/729,245 is a continuation-in-part of application No. 14/698,112, filed on Apr. 28, 2015, now abandoned.
(60) Provisional application No. 61/985,183, filed on Apr. 28, 2014.

(51) Int. Cl. A61B 90/14; A61B 90/11; A61B 90/30

(52) U.S. Cl. CPC A61B 90/14; A61B 90/30; A61B 90/11

(57) ABSTRACT

A neuro-surgical clamp device that has a semi-circular track that partially circles the patient's head. A C-clamp clamps securing to the patient's head, with the semi-circular track clamped to the C-clamp with a vice-clamp with at least one moving jaw. The C-clamp is coupled to a base bar though a series of three arms and four rotatable joints so that the semi-circular track can be tilted and rotated to any angle with respect to the patient's head. Instrument attaches can be clamped anywhere along the semi-circular track.

This is a continuation of application Ser. No. 16/289,131 filed Feb. 28, 2019 which was a continuation of application Ser. No. 15/729,245 filed Oct. 10, 2017 which was a continuation-in-part of application Ser. No. 14/698,112 filed Apr. 28, 2015 which claimed priority to U.S. Provisional Patent application No. 61/985,183 filed Apr. 28, 2014. Application Ser. Nos. 16/289,131, 15/729,245, 14/698,112 and 61/985,183 are hereby incorporated by reference in their entireties.

### BACKGROUND

### Field of the Invention

The present invention relates to clamps used in surgery to secure to the position between a piece of surgical equipment and a patient and more particularly to a neuro-surgical clamp used to secure and steady a retractor during neurosurgery.

### Description of the Prior Art

Clamps that are used in surgery to hold a piece of surgical equipment in proximity to a patient are known in the art. Position stability is particularly critical in a craniotomy procedure. However, prior art clamps do not adapt to attach to a head clamp or rest to easily support a retractor. Also, prior art clamps will not tilt to all desired angles.

Dinkier in U.S. Pat. No. 5,529,358 teaches a clamp that is equipped with a double arc for attaching instruments. It will not rotate and tilt to all angles.

Shuele in U.S. Published patent application number 2013/0081636 teaches a simple head clamp.

Sklar in U.S. Pat. No. 7,730,563 also teaches a neurological clamp that has tilt and angle difficulties typical of other prior art devices.

### SUMMARY OF THE INVENTION

The present invention relates to a clamp whose jaws are mounted on pivots. The clamp finds use in craniotomy procedures where a piece of the skull is removed to expose the brain. The clamp frame of the present invention can be attached to one or two armatures that circle the patient's head. The jaws hold the patient's skull fixed. In a particular embodiment (FIGS. 1-3), there are no supporting bars; rather, there is a pair of semi-circular (or other shaped) armatures that partially circle the patient's head and are attached to the frame of the clamp device. These allow the clamping or attachment of retractors and other instruments, all of which are held rigidly steady during the procedure. In another embodiment (FIGS. 4-5), there is a single arcuate armature that circles the head and hold devices. This embodiment can be rotated and tilted to any angle.

### DESCRIPTION OF THE FIGURES

Attention is now directed to several drawings that illustrate features of the present invention:

FIGS. 1A, 1B and 1C show side, top and isometric views of an embodiment of the clamp of the present invention.

FIG. 2 shows details of the use of the clamp of the present invention with armatures.

FIG. 3 shows details of an attachment between the clamp and the armature.

FIG. 4 shows a perspective view of an alternate embodiment of the present invention.

FIG. 5 shows a close up view of the vice-clamp in FIG. 4 that holds the accurate track to the C-clamp.

Several drawings and illustrations have been presented to aid in understanding the present invention. The scope of the present invention is not limited to what is shown in the figures.

### DESCRIPTION OF THE PREFERRED EMBODIMENT

The present invention relates to a double clamp whose jaws are mounted on pivots. The clamp finds use in craniotomy procedures where a piece of the skull is removed to expose the brain. The clamp frame of the present invention can be attached to armatures that circle the patient's head. The jaws hold the patient's skull fixed. There are no supporting bars; rather, there is a pair of semi-circular (or other shaped) armatures that partially circle the patient's head and are attached to the frame of the clamp device.

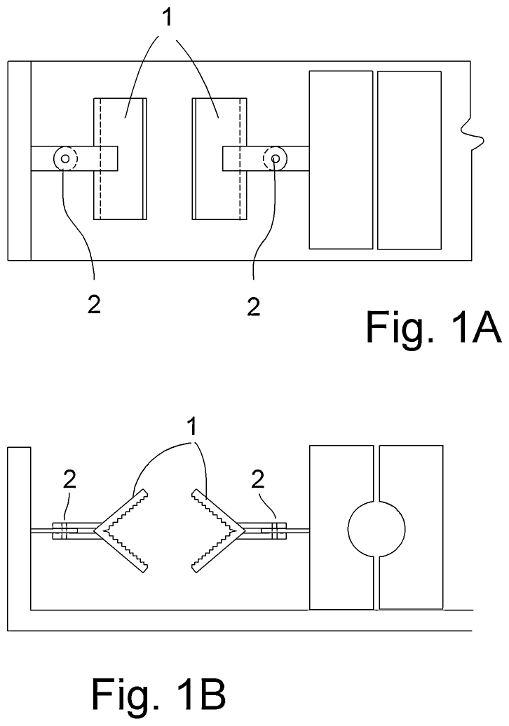

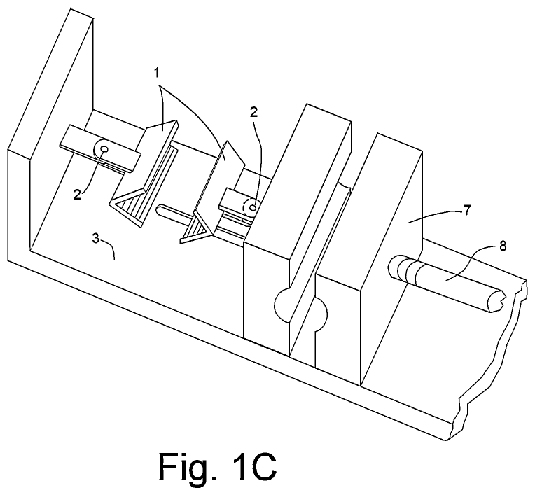

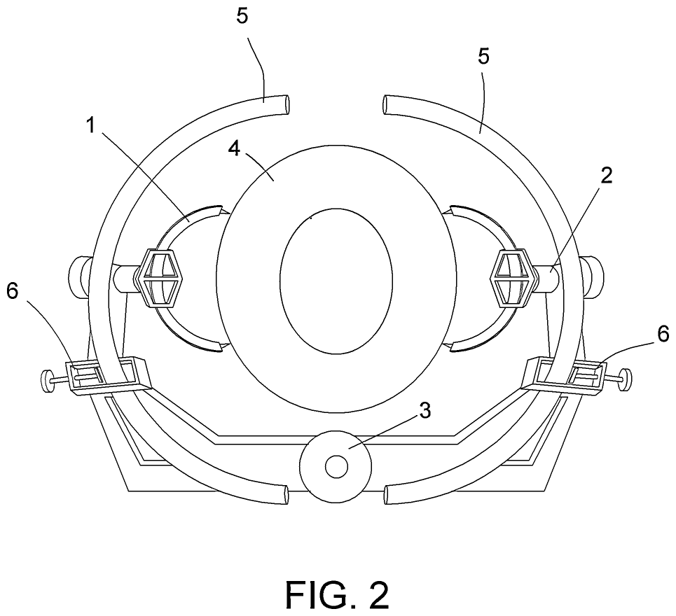

FIGS. 1A-1C show an embodiment of the clamp device. Jaws 1 are attached to a frame 3 by pivot joints 2. This allows the jaws 1 to freely pivot to attain the optimum position with respect to the patient's head 4 (FIG. 2). The pivot joints 2 are further attached to a frame 3 which can be wood, metal or any other rigid material. A movable block 7 can be screwed by a threaded rod 8 to tighten the jaws as shown in FIG. 1C. The jaws are adapted to contact the patient's head during surgery.

FIG. 2 shows an entire assembly schematically. The clamp frame 3 is attached to pair of semi-circular armatures 5 which partially encircle the patient's head 4. The armatures 5 can be metal or other rigid material. The attachment can be made through adjustable smaller clamps 6 or by any other attachment method that will allow secure attachment of that armatures 5 to the clamp frame 3. The armatures are used to hold surgical instruments. The armatures can be flat or other shape. The preferred shape is flat with a thickness smaller than their width. These typically take the shape of part of a semi-circle that is about twice the diameter of the patient's head. The armatures circle the head and provide a rigid support for the clamped or otherwise attached medical instruments. In an alternate embodiment, a single armature that is almost a full circle may be used.

The preferred material for the armatures is stainless steel; however, any rigid material may be used including other metals and hard plastics. The armatures must hold clamped medical instruments in place during a procedure without flexing or otherwise distorting or moving.

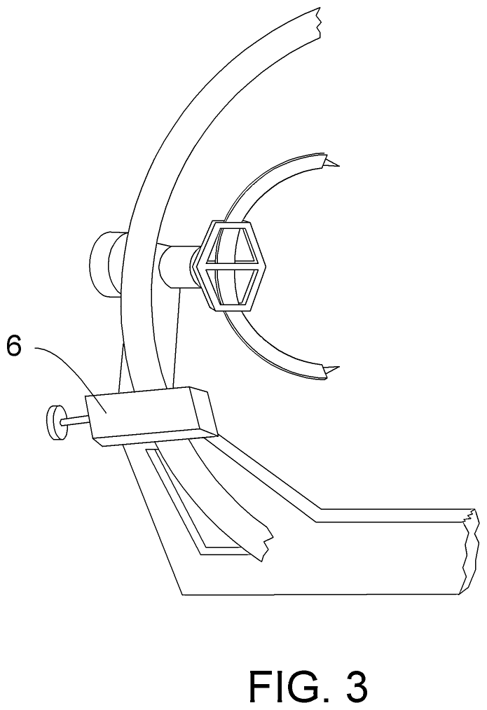

FIG. 3 shows a detail of an embodiment of a smaller removable clamp 6 used to attach the clamp unit of the invention to the armatures.

The present invention allows a retractor or other surgical instrument to be held in proximity to the patient's head in a totally fixed position typically during a craniotomy procedure. The instrument is held continuously steady during surgery. The retractor is typically clamped or otherwise attached to the armature during the procedure. The arrangement is simple enough that it is easy to set up and easy to rearrange.

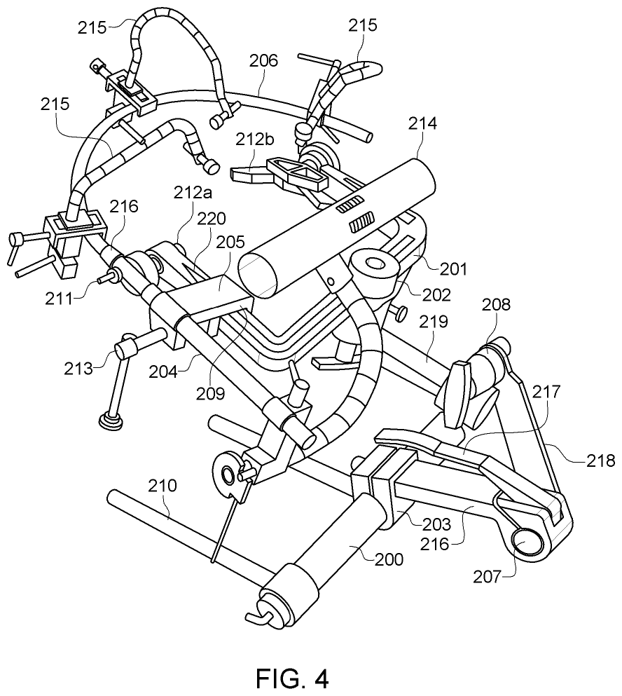

FIG. 4 shows a perspective view of an alternate embodiment of the present invention. A base bar 200 is held in place to a stable platform or attach (not shown) by a pair of legs 210. A pivot arm 216 clamps in a rotatable manner to the base bar 200. A rotatable clamp joint 203 allows the pivot arm 216 to rotate on the base bar 200. The pivot arm 216 extends to another rotatable joint 207 that is lever operated 217 allowing tilting. A flat arm or member 218 extends from that rotatable joint 207 up to yet another rotatable joint 208. That rotatable joint 208 is allows an arm 219 to rotate. At the end of the arm 219 is yet another rotatable joint in the form of a circular clamp 202 that holds a vice head C-clamp 201 by its center. The C-clamp 201 can slide in the circular clamp 202.

C-clamp 201 is used to clamp a human head in position for cranial surgery. It has a first head contact 212a and a second head contact 212b in opposition. The head contacts 212a, 212b are mounted on the ends of the C-clamp 201 and screw down to hold the patient's head. The two head contacts are different from each other with the point 212b having extended fingers. This is optional. Both head contacts can be identical.

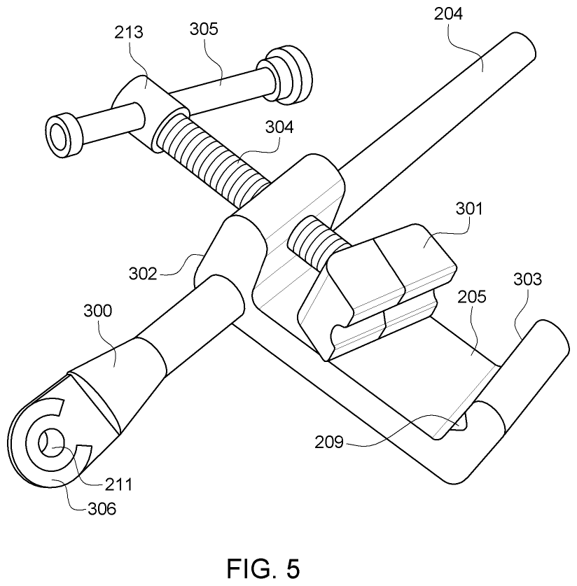

A vice-clamp 205 is used to clamp an arcuate track 206 onto one end 220 of the C-clamp 201 (the 212a end typically). The vice-clamp 205 allows a straight rod 204 to pass through a hole 302 in the vice-clamp (shown in more detail in FIG. 5). The vice-clamp 205 is tightened to the C-clamp end 220 with vice jaws tightened by a rotating shaft 213 a notch in the vice-clamp 205 allows the arm of the C-clamp to snugly fit and mate with the sliding jaw of the vice-clamp 205. The straight rod 204 is typically held in the vice-clamp 205 its clamping action. Alternatively, the straight rod 204 can be force-fit into the hole 302 in the vice-clamp 205.

The semi-circular accurate track 206 couples into the straight rod 204 in a swivel joint 211. This swivel joint 211 allows the accurate portion 206 to swivel with respect to the C-clamp 201. The swivel joint 211 can have a plurality of teeth 306 (shown in FIG. 5) to lock it in a fixed position.

The accurate track 206 allows a variety of clamped arms, instrument attaches 215 or other tools to be attached to it for use during surgery. Items like a light 214 can be placed on the straight rod 204, the accurate track 206 or elsewhere.

FIG. 3 shows a detail of the vice-clamp 205. The clamp base is C-shaped with a rotating screw 304 used to drive a single movable clamp jaw 301 toward a fixed jaw 303. The top of the rotating screw 213 has a sliding handle member 305. A slot 209 in the fixed jaw 303 allows holding of a rod such as might be found on a light (shown in FIG. 4). FIG. 3 shows the straight rod 204, which is on the end of the accurate track passing through a hole 302. The distal end 300 of the straight rod 204 is flared and terminates in a swivel joint 211. The swivel joint 211 has a series of ratchet teeth 306 that prevent slip. By loosening the a screw that passes through the swivel joint 211, the accurate track 206 (shown in FIG. 4) can be tilted to with respect to the straight rod 204 and hence, the rest of the apparatus. This swivel joint 211, along with the other adjustable joints of the device previously described allow the accurate track to take any position or angle desired.

Several drawings and illustrations have been presented to aid in understanding the present invention. One with skill in the art will realize that numerous changes and variations may be made without departing from the spirit of the invention. Each of these changes and variations is within the scope of the present invention.

I claim:

1. A neuro-surgical clamp device comprising:
a C-clamp (201) having a first end and a second end, the C-clamp rotatably attached to a first arm (219) through a first rotatable joint (202), the first arm rotatably attached to a second arm (218) through a second rotatable joint (208), the second arm rotatably attached to a third arm (216) through a third rotatable joint (207), the third arm rotatably attached to a base bar (200) through a fourth rotatable joint (203), the base bar constructed to be attached to a fixed support;
the C-clamp having a first head contact (212a) on the first end and a second contact (212b) on the second end, the first and second head contacts constructed to clamp a patients head during cranial surgery;
a half-circle semi-circular accurate track (206) attached to a straight rod (204) through a swivel joint (211), the swivel joint constructed to allow the accurate track to rotate with respect to the straight rod; the swivel joint having a plurality of teeth;
the semi-circular accurate track constructed to pass around the patient's head;
a vice-clamp (205) having a through-hole receiving the straight rod, the vice-clamp having at least one movable jaw, the vice-clamp clamped to the first end of the C-clamp;
the neuro-surgical clamp device being constructed to tilt and rotate to any angle with respect to the patient's head by adjustment of the swivel joint (211), the vice-clamp (205), the first rotatable joint (202), the second rotatable joint (208), the third rotatable joint (207) and the fourth rotatable joint (203);
a pair of legs (210) attached to the base bar (200) constructed to attach to the fixed support;
wherein, the first head contact has a single pointed contact, and the second head contact has a pair pointed contacts.

2. The neuro-surgical clamp device of claim 1 further comprising a light (214) attached to straight rod (204).

3. The neuro-surgical clamp device of claim 1 further comprising at least one instrument attach (215) removably clamped to the accurate track (206).

4. The neuro-surgical clamp device of claim 1 wherein the third rotatable joint (207) is lever-operated.

5. The neuro-surgical clamp device of claim 1 wherein the vice-clamp (205) has a fixed jaw and a movable jaw.

6. The neuro-surgical clamp device of claim 1 wherein the swivel joint (211) has a plurality of ratchet teeth (306).
